(19) United States
(12) Patent Application Publication
(10) Pub. No.: US 2015/0168284 A1
(43) Pub. Date: Jun. 18, 2015

(54) CAPILLARY VISCOMETER AND MULTISCALE PRESSURE DIFFERENTIAL MEASURING DEVICE

(71) Applicant: The United States of America, as represented by the Secretary, Dept. of Health and Human Services, Bethesda, MD (US)

(72) Inventors: Allen P. Minton, Bethesda, MD (US); Asaf Grupi, Nes-Ziona (IL)

(21) Appl. No.: 14/419,914

(22) PCT Filed: Aug. 20, 2013
(86) PCT No.: PCT/US13/55786

§ 371 Date: Feb. 5, 2015

Related U.S. Application Data
(60) Provisional application No. 61/691,209, filed on Aug. 20, 2012.

(51) Int. Cl. G01N 11/08; G01N 33/15

(52) U.S. Cl. CPC G01N 11/08; G01N 33/15

(57) ABSTRACT

The present subject matter provides a capillary viscometer for use in measuring concentration and shear dependence of the viscosity of macromolecular solutions. In one embodiment the device can automatically make serial dilutions of a single initial sample and record viscosity measurements across wide concentration ranges without changing samples. The device and associated methods can be used to rapidly and accurately assay solute stability and potentially solute-solute interactions in solutions of proteins and other macromolecules of pharmaceutical interest over a wide range of concentrations, including those corresponding to pharmaceutical formulations.

### CROSS REFERENCE TO RELATED APPLICATION

This application claims the benefit of U.S. Provisional Patent Application No. 61/691,209, filed Aug. 20, 2012, the disclosure of which is hereby incorporated by reference.

The present subject matter was made with U.S. government support. The U.S. government has certain rights in this subject matter.

### FIELD

The present subject matter relates generally to viscometers, i.e., devices for measuring viscosity of fluids, devices for measuring pressure differential, including automated devices for measuring the concentration and shear dependence of viscosity of dilute and concentrated macromolecular solutions and biologically-relevant samples.

### BACKGROUND

Current viscometer technology is limited in its ability to measure the rheological properties of biological or pharmaceutical samples. Published data for proteins depend on labor-intensive measurements of individual samples. In general, viscometers were originally created and adapted for measuring the rheological properties of industrial compositions which were usually non-aqueous and often highly viscous.

In contrast, measurement of the rheological properties of aqueous solutions of biologically-relevant macromolecules is a challenging task with potential implications for pharmaceutical formulations and delivery of concentrated therapeutics, as well as for characterization and understanding of the non-ideal behaviors of these complex macromolecules. In addition, measurement of concentration and shear dependent viscosity requires multiple sample runs with inherent difficulties of reproducible sample preparation. Further, biologically-relevant macromolecules are often isolated only after expensive laboratory experiments and which result in very small volumes of sample material. Moreover, relevant changes in the viscosity of aqueous solutions of biologically-relevant macromolecules can occur over relatively small ranges in concentration, which can be difficult to reproduce.

Thus, there remains a need in the art for a viscometer that can readily measure and compare the rheological properties of small volumes of aqueous solutions of biologically-relevant macromolecules under a variety of test conditions.

### BRIEF DESCRIPTION OF DRAWINGS

FIG. 1A shows a schematic view of one example of a viscometer system of the present subject matter.

FIG. 1B shows one example of a sample reservoir of the viscometer system of FIG. 1A having inlet and outlet tubing as well as a stirring device for stirring the sample, in this example a motorized stirring arm.

FIGS. 2A-2D are graphs of measured relative concentration plotted against calculated concentration thus validating the disclosed examples of automated dilution.

FIG. 3 is a graph of a voltage of the differential pressure sensor plotted against the flow rate, thus showing verification of proportionality of differential pressure and flow rate.

FIG. 4 is a graph of relative viscosities of glycerol and sucrose solutions measured at 20 C and plotted as a function of % w/w concentration.

FIG. 5 is a graph of a logarithm of relative viscosity of PEG solutions as a function of the mass fraction of PEG.

FIG. 6 is a graph of the intrinsic viscosity of PEG as a function of molecular weight as measured by the present method, with results from the literature plotted for comparison.

FIG. 7 is a graph of a logarithm of the relative viscosity of solutions of three proteins as a function of concentration in the low concentration limit, with the data points obtained via automated dilution according to the disclosed approach and the curves representing calculations as set forth in Table 3.

FIG. 8 is a graph of a logarithm of relative viscosity as a function of hemoglobin concentration.

FIG. 9A is a graph of the viscosity of hemoglobin plotted as a function of concentration as measured by the present method, together with the calculated best fit of a theoretical model.

FIG. 9B is a graph of the fit residuals from FIG. 9A.

FIG. 10 is a drawing of a screen display showing initialization of a master program.

FIG. 11 is a drawing of one example of a user interface for use with the present subject matter which comprises three main parts.

FIG. 12 is a drawing showing an alternative in-line tubing and series pressure sensor configuration.

FIG. 13 is a drawing showing an expanded view of an in-line tubing and parallel pressure sensor configuration.

FIG. 14 is a graph of shear rate plotted against viscosity in the low range showing the effects of increasing backpressures.

FIG. 15 is a graph of shear rate plotted against viscosity in the midrange showing the effects of different backpressures.

FIG. 16 is a graph of shear rate plotted against viscosity showing the increase in flow rate and shear rate for a larger syringe.

FIG. 17 is a graph of shear rate plotted against viscosity in the high range showing the effects of different backpressures.

FIG. 18 is a graph of apparent viscosity plotted against apparent shear rate for two different types of fluids and using two different pressure hose configurations.

### SUMMARY

The present subject matter relates to a device that can readily measure and compare the rheological properties of small volumes of aqueous solutions of biologically-relevant macromolecules under a variety of test conditions, and optionally with automated in-line serial dilutions of a single sample.

In one embodiment, the subject matter provides a viscometer device capable of automated in-line sample dilution and automated viscosity data acquisition over a range of dilutions. Another embodiment provides an automated viscometer having an in-line dilution capability comprising a closed-circuit pressure tubing system that a viscosity sample can flow through; at least one in-line automated pump for moving the sample through the closed-circuit tubing system; at least one in-line automated multiple distribution valve for adding liquid or sample to the tubing system, removing liquid or sample from the tubing system, or combinations thereof; an in-line pressure test-zone tubing section; and at least one pressure differential sensor for measuring the change in pressure across the pressure test-zone tubing section. A further embodiment provides a viscometer comprising a closed-circuit pressure tubing system that a viscosity sample can flow through; at least one in-line pump for moving the sample through the tubing system; at least one in-line distribution valve connected to the at least one in-line pump for adding liquid or sample to the tubing system, removing liquid or sample from the tubing system, or combinations thereof; at least one in-line sample reservoir; at least one in-line pressure test-zone tubing section; and at least one pressure differential sensor for measuring the change in pressure across the pressure test-zone tubing section.

The subject matter also provides a device capable of automated in-line sample dilution and automated data acquisition over a range of dilutions for any physical measurement of a sample, not limited to viscosity (such as light scattering, light absorbance, fluorescence, NMR, ESR, Raman spectra, etc.) In addition, the subject matter provides a multiscale pressure differential sensor device comprising two or more pressure differential sensors for measuring multiscale pressure differentials with an accuracy ranging in one non-limiting example from about 0.2% to about 2.0% across a broad range of pressures (one example, ranging from about 1 psi to about 350 psi.)

In certain embodiments, the present subject matter includes a device for thermostatically controlling the sample temperature, non-limiting examples include a liquid immersion bath or thermoelectric device, and may optionally include a coiled or other-shaped portion of the tubing system to assist with temperature control of the sample in the tubing system. In one embodiment, a coil of stainless steel tubing is used to equilibrate the sample temperature in the thermostatic device as the sample flows through the coil.

In yet another embodiment, the present subject matter provides a device and method for measuring the concentration and shear dependence of a single sample of a small volume of a biologically-relevant aqueous sample. Furthermore, some embodiments provide for an automated method wherein a single small volume aqueous sample is inserted into a device of the present subject matter and concentration and sheer dependent viscosity is measured in an automated process wherein serial dilution of the sample and sequential pressure differential-based viscosity measurements of each dilution can be made quickly and easily without stopping data acquisition or changing samples.

In a further embodiment, the present subject matter provides a method for measuring viscosity of a sample accurately over a wide range of viscosity through the use of two or more differential pressure sensors with similar or different sensitivity ranges and placed in parallel, series, or series-parallel with the tubing through which the sample flows and across which the differential pressure is developed. In some embodiments, differential pressure sensors detect differential pressure between two flow points by means of hydraulic tubing through which the sample does not flow. Shutoff valves may be used to protect high sensitivity sensors against possibly damaging overpressure, and such valves may be opened manually or automatically when the differential pressure measured by a higher pressure range sensor (lower signal to noise sensitivity) decreases to values compatible with safe operation of a lower pressure range sensor (higher signal to noise sensitivity).

In still another embodiment, viscosity may be measured as a function of concentration and shear dependence comprising: (a) injecting a sample solution to be tested at a known concentration and flow rate through the pressure tubing system; (b) recording measurements from at least one low sensitivity sensor and, optionally, from at least one high sensitivity sensor; (c) collecting the sample in an in-line sample reservoir; (d) diluting the sample in the sample reservoir by (i) removing a predefined amount of sample from the sample reservoir through a distribution valve, and (ii) adding a predefined amount of diluent provided through a distribution valve; (e) circulating the diluted sample solution through the pressure tubing system; (g) recording measurements from the differential pressure sensors; and (h) repeating parts (c), (d), (e), (f), and (g) until the minimum desired dilution of the sample is tested. If more than one flow rate is required then parts (a) and (c) are repeated for each flow rate.

### DEFINITIONS

For the purposes of this subject matter, the following terms will have the following meanings unless specifically stated otherwise:

The term “viscosity” means a physical property that characterizes the flow resistance of a fluid; it is a measure of the internal friction of a fluid where the friction becomes apparent when a layer of a fluid is made to move in relation to another layer; it is the resistance experienced by one portion of a material moving over another portion of material. A “viscometer” is a device for measuring viscosity of a fluid sample. The term “fluid” refers to a material in the fluid state or a material that is capable of flowing through a tubing system of a device of the present subject matter, and fluids may optionally comprise materials not necessarily in a fluid state, non-limiting examples may include nanoparticles, solid particles, a gel-state, colloids, liquid crystals, petrochemicals, greases, oils, etc. The phrase “viscosity sample” means any fluid that is capable of flowing through a tubing system of a device of the present subject matter and which viscosity dependent shear can be measured. Non-limiting examples of viscosity samples that can be measured with one or more devices described herein include biological fluids, aqueous fluids, non-aqueous fluids, petrochemical fluids, fluids comprising biological materials, chemicals, pharmaceuticals, excipients, salts, solvents, and combinations thereof.

The phrase “pressure tubing” as used herein means tubing that can withstand pressure ranges normally used in capillary viscometer devices without collapsing, deforming, or otherwise failing to maintain its shape, internal volume, and internal sample flow characteristics. In general, “pressure tubing” allows for flexible adjustment of the instrument setup to different ranges of viscosities and/or shear rates. The tube inner diameter is the most important parameter in determining the system measurement range as the differential pressure is strongly dependent on the tube radius, i.e., Pαr⁻⁴.

The phrase “pressure tubing system” and “tubing system” as used herein are interchangeable and refer to the tubing selected and interconnected for use in a device of the present subject matter. In some embodiments, the pressure tubing system is a “closed-circuit.” “Closed-circuit” as used herein refers to a tubing system which can be closed to the outside environment or outside pressure and placed under its own independent pressure within the closed-circuit. In some embodiments, the closed-circuit pressure tubing system may be temporarily opened to the outside environment or outside pressures during a sample run, such as, for example, when sample, solvent, or waste is moved into or out of the tubing system, or when measuring pressure differential compared to atmospheric pressure. “In-line” as used herein refers to a component or device that is connected to a pressure tubing system of the present subject matter such that the in-line component or device may act on the sample or contact the sample flowing through the tubing system, non-limiting examples of an in-line component may include a pump, a valve, a sensor, a reservoir, a flow cell, etc.

A wide variety of different pressure tubing may be used in embodiments of the present subject matter including non-limiting examples such as plastic, glass, polymer and metal. For example, a wide selection of PEEK tubings, with varying diameters and lengths, is available. The length of the tube, L, is linearly proportional to the pressure differential. For very viscous (high viscosity) solutions, the volume of the tubing used in the system may require using a larger cross-sectional area and thus larger sample volume, which might not be available or convenient for biologically-relevant samples prepared from expensive or small scale biological sources or experiments. In one non-limiting example, a total pressure tubing system volume comprises both 0.02″ and 0.03″ inner-diameter tubing and have a total internal tubing volume 480 μl. Replacing all of the tubing with 0.04″ inner-diameter tubing will increase the total tubing system volume to about 900 μl and the total sample volume to about 1200-1500 μl. Various diameters and lengths of tubing can be selected to design a variety of final total volumes for tubing systems of the present subject matter.

The phrase “pressure test-zone tubing section” is used herein to refer to at least one predefined section of pressure tubing in the tubing system over which length the pressure differential will be measured for calculating viscosity measurements. The pressure test-zone tubing section has a defined length and radius of cross-sectional area which are used along with pressure differential measurements in calculating viscosity measurements. The pressure test zone is not limited to cylindrical tubings but could include any orifice or cross-sectional shape that provides resistance to flow.

The phrase “flow rate” as used herein refers to the rate that the sample flows into or through a pump, injection device, or pressure tubing system. A pump or injection device allows for a wide range of flow rates through the tubing system, and the flow rate may selected and/or adjusted by selecting the syringe or injector volume and the pump rate. In one embodiment, the flow rate spans three orders of magnitude of shear rate in a single experiment. In one non-limiting example: a solution of viscosity of 50 cP is: D=0.01″, L=10 cm, sensors=250 PSI and 30 PSI, for 0.42 ml/min, Shear rate=73 s-1 and the pressure is 1 psi for 15 ml/min, Shear rate is 2591 s-1 and the pressure is 250 PSI.

The phrases “differential pressure” or “pressure differential” as used herein are interchangeable and refer to the difference in pressure between two selected points in a pressure tubing system. Any type of pressure differential sensor may be optionally used with the present subject matter, non-limiting examples include commercially-available pressure differential sensors. In one embodiment, the pressure differential sensor comprises a device with two hydraulic fluid compartments separated by a pressure sensitive membrane that generates a signal proportional to the pressure differential across the membrane and the sensor then generates a proportional electrical signal, and the two hydraulic fluid compartments are connected to two hydraulic fluid lines that connect to the two locations on the pressure tubing system where pressure differential measurement is desired. In one embodiment, the pressure differential measurement is taken across the length of the pressure test-zone tubing section by attaching the hydraulic lines of a pressure differential sensor near the beginning and end of the pressure test-zone tubing section. In general, when using such an embodiment, the inner volume and pressure of the hydraulic lines of a pressure differential sensor are in open contact with the inner volume and pressure of the pressure tubing system of the present subject matter. In such a configuration, it is noted that the sample being tested in the tubing system does not generally enter the pressure sensor hydraulic lines because of the smaller diameter of the hydraulic lines and passive barriers, and because there is no fluid flow at all through the sensor because of the sensor membrane barrier between both hydraulic lines.

The phrases “sensor range” or “pressure sensor range” as used herein are interchangeable and refer to the range of pressures that a particular pressure sensor is capable of measuring. In some embodiments, the pressure sensor device may have “multiscale pressure” sensitivity, which refers to a range of pressures that range over multiple scales, non-limiting examples of the present subject matter include ranges from about 1 cP to 1000 cP. In one embodiment, the pressure sensor device may comprise two or more pressure differential sensors with different pressure ranges are which are connected in parallel, in series, or in series-parallel to the pressure tubing. In another embodiment, only one pressure differential sensor is sufficient to get an accurate reading of a gradient ranging from low (about 1-2 cP) to mid-viscosity (about 1-50 cP) solutions. In a further embodiment, where a viscosity range in a concentration gradient is large, a first pressure differential sensor (with a higher pressure range) is used to measure the high viscosity region and a second pressure differential sensor (with a lower pressure range) is used to measure the low viscosity region. In other embodiments, a low viscosity sensor can be protected from damage caused by over pressure using two or more valves, for example, two valves can be placed on each side of the second sensor, wherein both valves are closed at high pressure, and both are opened when the pressure is in the range of the high sensitivity low viscosity sensor. The sensor output may be optionally connected to a data acquisition module. A non-limiting example of an instrumental setup that allows the measurement of the viscosity of a solution under a concentration gradient with viscosities in the range of 1-1000 cP would be: D=0.02″, L=10 cm, Flow rate=0.2 ml/min, Sensors: 30 PSI, 5 PSI.

The term “pump” as used herein refers to a device for moving a fluid, including moving a fluid through a pressure tubing system of the present subject matter. The pump of the present subject matter may be manual or automated. In one embodiment, the pump is selected from a manual syringe and an automated syringe pump.

The term “distribution valve” as used herein refers to a component for opening, closing, or diverting the flow of a fluid through a chamber or tubing, including opening, closing, or diverting the flow of a fluid through a pressure tubing system of the present subject matter. In one embodiment of the present subject matter, the distribution valve is a multiple distribution valve having multiple ports for opening, closing or diverting the flow of fluid between multiple inlets, outlets, and/or tubing. In another embodiment, the pump may be integrated with at least one distribution valve, and the pump and valve(s) may be optionally automated and programmable. In a further embodiment, the pump is integrated with a syringe and a multiple distribution valve having at least four inlets/outlets, and the pump, syringe, and distribution valves are all automated and programmable.

The pump and valve pressure limit can be important variables in the system. For example, if a valve has a pressure limit of 100 PSI, the total pressure in the tubing system should be <100 PSI to avoid reaching the designated pressure limit. However, in some embodiments, the tubing system can be designed so that all the tubing, except for the separate pressure test-zone tubing section, has negligible pressure (such as, by using 0.04″ diameter tubing) and only the pressure test-zone tubing section can be considered as providing the main source of pressure buildup on the valve. However, when using 0.04″ diameter tubing, the total sample volume will be increased as compared to narrower diameter tubing.

By using pump and valve combinations with different pressure limits, the range of shear rates can be customized for some embodiments. For example, using a pump and valve combination with higher pressure limits will extend the range of measurement and applications.

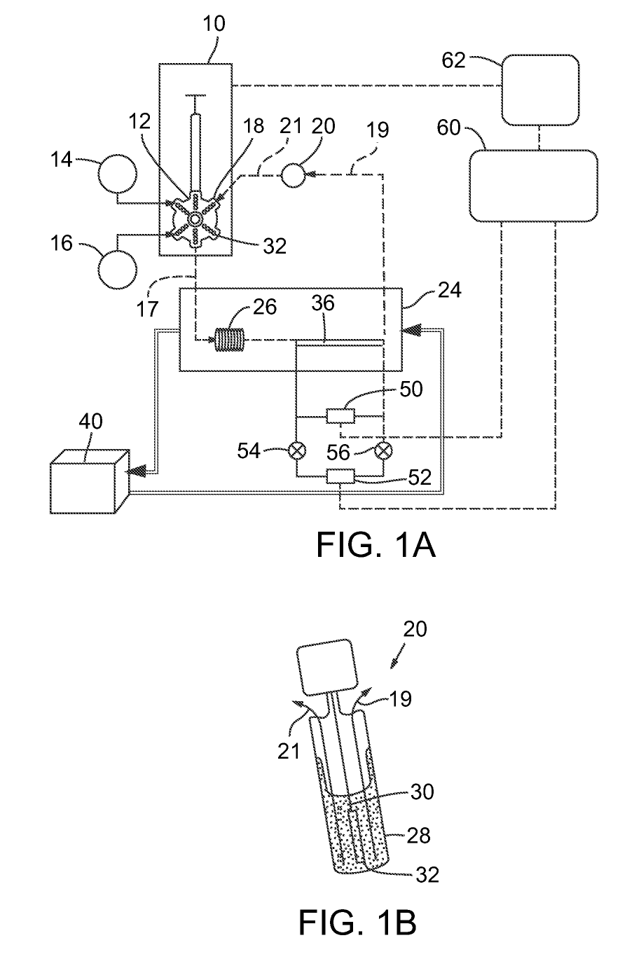

The phrase “calculation of the sample volume” as used herein means determining the volume of sample in a specific component, such as, for example, total volume of sample in the pressure tubing system or total volume of sample in the pressure test-zone tubing section. The total volume of a sample in one embodiment equals the sum of the volume of the tubing in the closed path (FIG. 1, dashed lines) and the sample volume in the in-line sample reservoir. In one embodiment, the volume of the tubing may be calculated by, first, filling the tubing with water, and then removing the fluid of each tubing, (i.e., (1) from the loop outlet and (2) from the solution inlet) using a volumetric syringe and reading the volume on the syringe measurement tick marks. This can be a one-time procedure for some embodiments where loop segments are not replaced. As loop segments are replaced, their volume can be predetermined for accurate calculation of total tubing system volume. The sample volume in an in-line sample reservoir is then the total desired system volume less the tubing volume.

The phrase “sample reservoir” as used herein means an area where a viscosity sample being measured is stored, and the reservoir is connected to the pressure tubing system of the present subject matter. The sample reservoir may be connected as an in-line reservoir in the pressure tubing system or connected to the pressure tubing system by way of a distribution valve. The sample reservoir may be located in any location of the pressure tubing system, provided that it is not located in the pressure test-zone section. In some embodiments, the sample reservoir is located in-line and in close proximity to a distribution valve that functions to add sample or diluent to the sample reservoir or tubing system and/or to remove sample or diluent from the sample reservoir or tubing system. In general, samples being measured should be well-mixed especially when samples are being diluted or concentrated being measurements. Samples can be mixed while present in the device by any reasonable means possible. Non-limiting examples of mixing samples in the device include mixing sample and diluent in the sample reservoir with physical agitation. Another example provides that the sample can be mixed by cycling the mixture through the pressure tubing system (such as one or more times through the tubing system) without the need for an extraneous means for mixing.

The present subject matter relates to a viscometer device and method for quickly and easily measuring the rheological properties of small volumes of aqueous solutions of biologically-relevant macromolecules, including measuring the concentration and sheer dependent viscosity. In addition, the present subject matter provides a viscometer device that can measure concentration and sheer dependent viscosity in a single sample by using a reproducible automated serial dilution and differential pressure measurement routine on a single small volume of sample. Thus, the present subject matter provides a viscometer and method that are useful for measuring and comparing the rheological properties of small volumes of aqueous solutions of biologically-relevant macromolecules.

Table 1 shows a comparison of features of one example of a visocometer of the present subject matter with those of the commercially available Rheosence VROC viscometer/rheometer.

TABLE 1

| | | A commercially available |
|---|---|---|
| | | viscometer (VROC - |
| | One example of a viscometer | Viscometer/Rheometer on |
| | of the present subject matter | chip, Rheosense) |
| Automated dilution | Yes | No |
| Sample volume | 750 ul (equivalent to ~30 | 50 ul per sample (for a narrow |
| | ul for a gradient of 26 | range of viscosity and shear) |
| | dilution steps) | |
| Variation in shear rate | Can cover a wide range in a | Highly dependent on sample |
| | single experiment | volume |
| Data analysis | Viscosity is a linear function | Viscosity is a nonlinear |
| | of measured signal | function of measured signal, |
| | | requiring extensive calibration |

Further, in some embodiments the present subject matter provides a viscometer/rheometer device that can automatically make measurements of viscosity over a large range of compositions and shear rates under automated program control. In one embodiment, the device may be constructed primarily from inexpensive off-the shelf components and the sample volume requirements may be generally much smaller than most presently available commercial devices. Also, maintenance of a device of the present subject matter may be generally simple and inexpensive since maintenance requires only simple replacement of relatively inexpensive commercially available capillary tubing and pressure sensors. Moreover, the device's range of applicability may be extended or customized by selecting replacement capillary tubing and pressure sensors having technical specifications matching a particular range of desired utility.

### Example 1

### Description and Operation of an Apparatus

In one embodiment, the apparatus consists of several parts, each of which is shown in FIG. 1A. A programmable single-syringe pump 10 (Hamilton, PSD/8) is connected to a 6-way distribution valve 12 which controls fluid flow and source/destination of fluid flow. The distribution valve 12 has ports that are connected to a diluent reservoir 14 containing solvent, a reservoir 16 for collection and recovery of the sample removed at each dilution step, an inlet 18 through which the syringe 10 is loaded with solution from the solution vial 20, a pressure test-zone tubing section 36 that leads back into the solution vial 20, and an optional reference solution (via open valve port 32). Following each dilution step, and as shown in FIG. 1B, the solution is mixed by an overhead stirrer 30 (Spectrocell) fitted to the top of a cylindrical vial 28, equipped with a custom made metal paddle 32. The solution vial 28 is tilted to ensure that all contents may be extracted via the outlet tubing 21.

During operation, solution flows from the syringe 10 through PEEK™ (polyetheretherketone) polymer tubing 17 into a sealed polycarbonate water bath 24 used to maintain temperature kept constant by a water pump 40. The solution then flows through a 100 μl, 0.02″ stainless steel tube 26 to ensure thermal equilibration with the water bath 24, and then through the pressure test-zone tubing section 36 formed as a capillary constructed of PEEK™ tubing that is connected at both ends through tee fittings (Upchurch) to two Omega PX-26 series piezoelectric differential pressure sensors 50, 52 mounted in parallel. Two manual on/off valves 54, 56 are positioned between the two sensors for sensor selection. After passing through the pressure test-zone tubing section 36, the solution returns to the solution vial 20. A model 6211 National Instruments data acquisition and digital control module 60 connected to a Windows PC 62 is used to collect analog data from the pressure sensors. The syringe pump 10 and distribution valve 12 are controlled directly by the PC 62 through an RS232 serial interface.

The total backpressure during injection caused by all of the tubing in the system is limited to the maximum backpressure allowed for either the pump or the distribution valve. The backpressure can be reduced by choosing larger tubing diameters. Larger tubing diameters do require, however, use of a larger sample size.

For example, for a system in which the backpressure limit is 100 psi (total system backpressure should be less than 100 psi), the effect of tubing diameter and length on sample volume is described in the following table.

| | Total sample volume (ul) | |
|---|---|---|
| | (tubing volume + valve | Maximum allowed |
| | dead volume + 250 ul | pressure in pressure |
| System tubings scheme | sample in vial) | tubing (psi) |
| D = 0.02″, L = 100 cm | 553 | 59.0 |
| D = 0.02″ L = 60 cm | 654.2 | 68.1 |
| D = 0.03″ L = 40 cm | | |
| D = 0.03″ L = 100 cm | 806 | 88.0 |
| D = 0.04″ L = 100 cm | 1161 | 95.9 |

In one embodiment, all operations of the apparatus, as well as data storage, processing, and analysis as described below, are controlled by user-written scripts and functions in MATLAB (R2006b, Mathworks). A detailed description of the software is provided in the supplementary information.

### Description of Data Processing and Analysis

Measurement of viscosity and relative viscosity. The differential pressure between two ends of a cylindrical capillary of length l and radius r through which fluid of viscosity η is flowing at rate ν is given by Poiseuille's law (Tanford 1961):

$$Δ P = \frac{8 vl η}{π {r}^{4}} \qquad [ 1 ]$$

In one embodiment, the differential pressure sensors used in the instrument described here produce a DC voltage, denoted by S_{raw}, given by

S_{raw}=αΔP+S_{raw}^{o} [2]

where S_{raw}^{o} denotes an offset voltage measured in the absence of a pressure differential and α is a proportionality constant calculated from a given sensor's specifications as α=ΔPₘₐₓ/Sₘₐₓ, where ΔPₘₐₓ is the high end limit of the sensor range and Sₘₐₓ is the voltage produced at ΔPₘₐₓ. The values of α and S_{raw}^{o} for a given sensor, and the accuracy of equation [2] are determined by measurement of S_{raw} as a function of flow rate ν for a Newtonian fluid of known viscosity. The sensors utilized have been found to be accurate to within <0.5% of their full range. We may thus utilize the values of α and S_{raw}^{o} so determined to calculate the differential pressure according to

ΔP=(S_{raw}−S_{raw}^{o})/α

with known precision. In order to ensure that ΔP can be measured over a wide range of pressures with optimal accuracy and precision, the instrument is equipped with two pressure sensors in parallel whose sensitivities differ by a factor of usually 5 to 30. The default sensor is the low sensitivity sensor, which will not be damaged by differential pressures that might damage the high sensitivity sensor. However, when the differential pressure drops below a pre-defined limit deemed safe for the high sensitivity sensor, the controlling program may signal the user to open valves that activate the high sensitivity sensor, thus providing higher resolution pressure data at low pressures. Given an accurate measurement of ΔP, the absolute and relative viscosities can be measured according to

$$η = \frac{π {r}^{4}}{8 vl} Δ P \qquad [ 4 ]$$

and the relative viscosity of a solution containing w/v concentration w of solute according to

$${η}_{r} ( w ) ≡ \frac{η ( w )}{{η}_{0}} = \frac{Δ P ( w )}{Δ {P}_{0}} \qquad [ 5 ]$$

where η₀ and ΔP₀ respectively denote the viscosity and differential pressure of solvent at the same flow rate.

Calculation of Intrinsic Viscosity.

The intrinsic viscosity of a solute is defined as

$$[ η ] ≡ {Lim}_{w \to 0} \frac{η ( w ) - {η}_{0}}{{η}_{0} w} \qquad [ 6 ]$$

Hence we may identify the intrinsic viscosity as the coefficient of the linear term in an expansion of either ηᵣ or ln ηᵣ in powers of w:

ηᵣ=1+[η]w+Bw²+ . . . [7]

ln ηᵣ=[η]w+Cw²+ . . . [8]

and may thus be evaluated by fitting a polynomial to the measured dependence of either ηᵣ or ln ηᵣ upon w at limiting low values of w.

### Materials

Protein Samples for Intrinsic Viscosity Measurements:

Fibrinogen from bovine plasma (Sigma, F8630) was prepared by dissolving 80 mg in 10 ml saline solution (0.9% NaCl) at 37 C followed by dialysis in a 10 kD dialysis cassette against 40 mM PBS, 1=0.45. The solution was concentrated to 25 mg/ml with a 10 kD ultrafiltration device (Amicon, Millipore). BSA (Sigma, A1900) was dissolved and dialyzed against 23 mM Sodium Acetate buffer pH 5, 0.2M NaCl. Ovomucoid (Warthington, 3086) was dissolved and dialyzed against 150 mM sodium acetate buffer, pH 4.65. Ovomucoid and BSA were filtered with a 0.1 um Anotop syringe filter (Whatman). All protein samples were centrifuged for 30 minutes at 50000G, 20 C to remove large aggregates and dissolved gases. Protein samples were measured without further purification.

Concentrated Hemoglobin:

whole blood was diluted with isotonic solution of 0.9% NaCl in a 1:2.5 w/w ratio, respectively. The solution was washed three times by pelleting the blood cells by centrifugation (15 min, 12000×g) and resuspending with saline solution. Protein was extracted by resuspension of the cell paste with ice-cold water under vigorous stirring for 45 minutes on ice. Cell debris was removed from protein extract by centrifugation for 30 minutes at 12000 g. The supernatant was removed and kept at 4 C. SEC analysis was used to estimate protein solution purity. Hb protein molecules were converted to the cyanmet form as previously decribed (Crosby and Houchin 1957). Hb concentration was determined by absorbance at 523 nm (Snell and Marini 1988) in order to avoid miscalculation of protein concentration due to partial cyanmet conversion. Hb was concentrated to 325 mg/ml by ultrafiltration with 10 kD membrenes.

Polyethylene Glycol (PEG):

PEG fractions of five different average molecular weights (200, 400, 600 and 2000D from Sigma, 1000D from Fluka) were used in one of the viscosity experiments. Samples were prepared by dissolving weighed PEG in weighed water followed by overnight tilting for complete dissolution. All samples were used without further purification.

Sucrose and Glycerol:

Ultrapure Sucrose (Invitrogen, cat#: 15503-022) and Glycerol (Sigma, cat#: 15523) were used. A 90% w/w solution of Glycerol was prepared by mixing ultrapure H₂O with glycerol, and a 70% Sucrose w/w solution was prepared as described previously (Quintas, Brandao et al. 2006). Sucrose and glycerol concentrations were determined from via differential refractometry as previously published (Lide 2004).

### Results

Validation of Dilution Protocol.

A sample solution is diluted by removing an aliquot of the solution to the waste container followed by the addition of an equal volume of diluent to the sample vial. The volumes are precalculated by the software, given the total solution volume and the desired fractional extent of dilution per increment of dilution. This approach keeps the total solution volume constant in the absence of significant mixing non-additivity. When dilution results in a significant change in solution density, a correction must be made in order to obtain the actual mass present at each dilution step. An example of a dilution sequence is provided in Table 2, which shows an essential requirement for a successful dilution step is that the sample will be completely mixed in the tubing and sample vial. This is accomplished by means of continuous stirring of the sample with an overhead mixer and by washing the closed loop (with the sample vial to vial) which takes about three tubing volumes.

TABLE 2
Sample dilution sequence.
A linear gradient of concentration was obtained, with the final dilution
resulting in a solution with 10% of the initial concentration.
This gradient was obtained in 10% increments as follows:

| | Fraction of | Resulting w/v | Example: Volumes |
|---|---|---|---|
| | solution volume to | concentration in | removed/added |
| Dilution | be removed and | % of original | for a 1000 ul |
| step # | replaced by solvent | concentration | solution volume |
| 1 | 1/10 | 90 | 100 |
| 2 | 1/9 | 80 | 111 |
| 3 | ⅛ | 70 | 125 |
| 4 | 1/7 | 60 | 143 |
| 5 | ⅙ | 50 | 167 |
| 6 | ⅕ | 40 | 200 |
| 7 | ¼ | 30 | 250 |
| 8 | ⅓ | 20 | 333 |
| 9 | ½ | 10 | 500 |

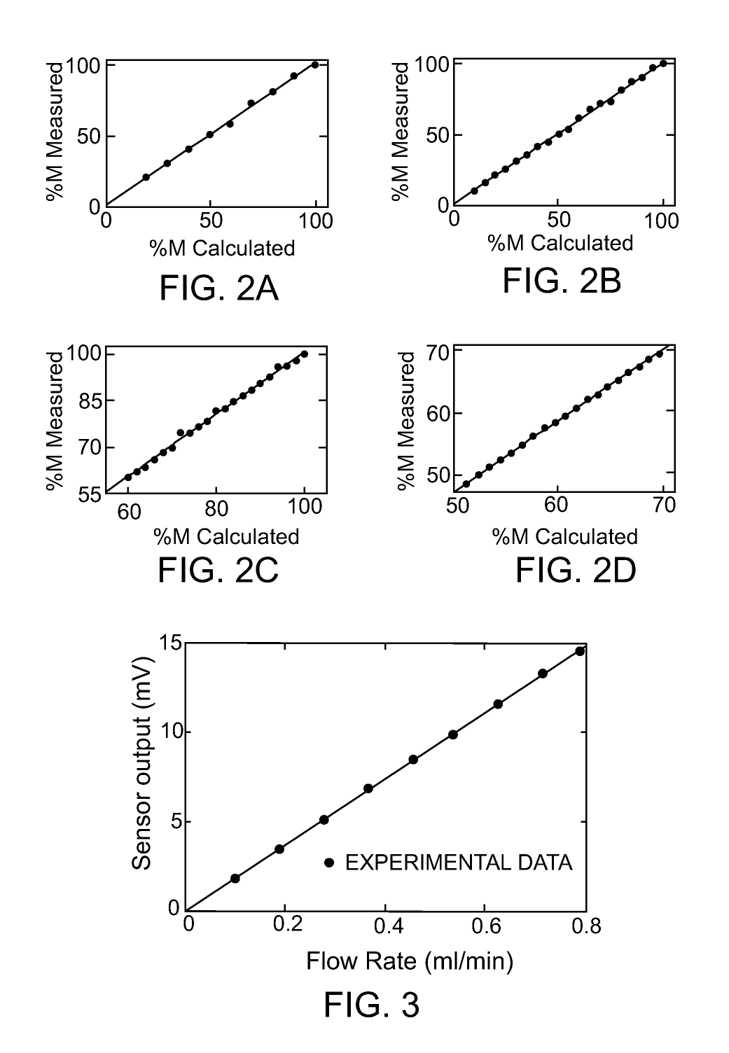

In order to evaluate the accuracy of dilution at low viscosity, 1 ml sample solutions of 40 uM fluorescein in PBS pH 7.4, were diluted at 10%, 5% and 2% Volume steps. The volume removed at each dilution step was collected and the absorbance of each sample was measured at 470 nm. The absorbance of all samples from a single gradient were normalized to the absorbance of the solution prior to dilution. The accuracy of dilution was evaluated by plotting the calculated relative concentration vs. the measured relative concentration by absorbance. The results shown in FIGS. 2A-C indicate that the calculated dilution is accurate to within the precision of measurement. The accuracy of dilution of high viscosity solutions was also checked. A 1 ml sample of 70.6% W/W sucrose was diluted at 2% steps by volume for 20 steps. The solution volume removed at each dilution step were collected and the refractive index was measured. Published data from tables of the refractive index dependence on sucrose concentration was fitted to a polynomial in order to calculate the concentration of the collected samples (Lide 2004). The relative concentration calculated, taking density effects into account, is plotted against the measured relative concentration in FIG. 2D (the solid circles represent experimental data). A linear fit of the calculated solute concentrations (% M Calculated, solid line) to the experimentally determined concentrations (% M Measured, solid circles) validates the dilution apparatus with a slope of 1.002-1.012 and a Y-intercept of 0.063-0.15. The results again show that the dilution and mixing is efficient and accurate for a solution, the initial viscosity of which is over 350 cP at room temperature.

Verification of Proportionality of Sensor Response, Differential Pressure and Flow Rate.

In FIG. 3, a sensor output (mV) is plotted as a function of the flow rate of a 50% weight fraction glycerol solution. Sensor output depends linearly upon flow rate as predicted for a Newtonian fluid by equations [1] and [2].

Viscosity of Concentrated Glycerol and Sucrose Solutions.

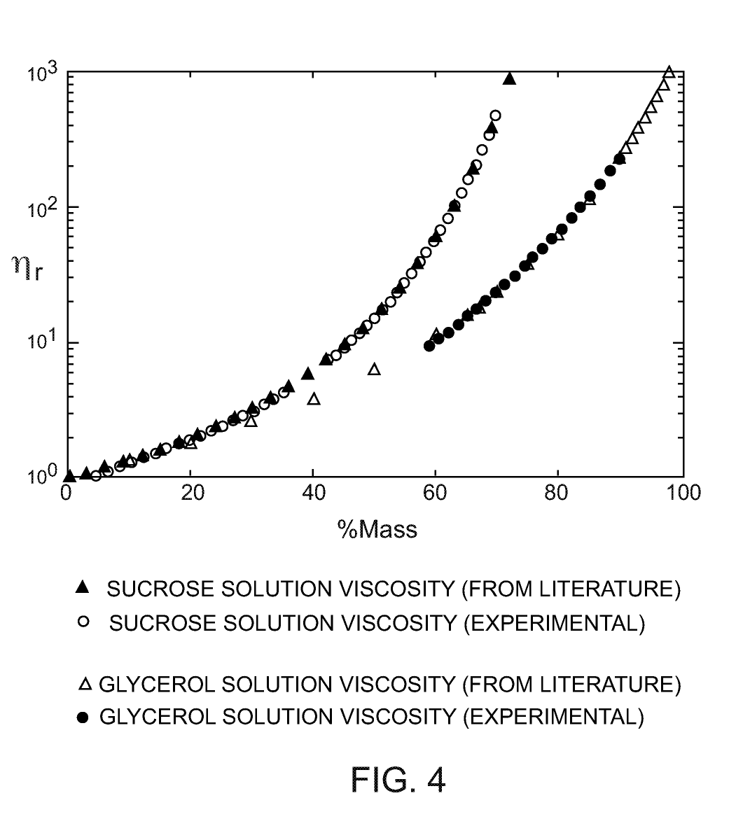

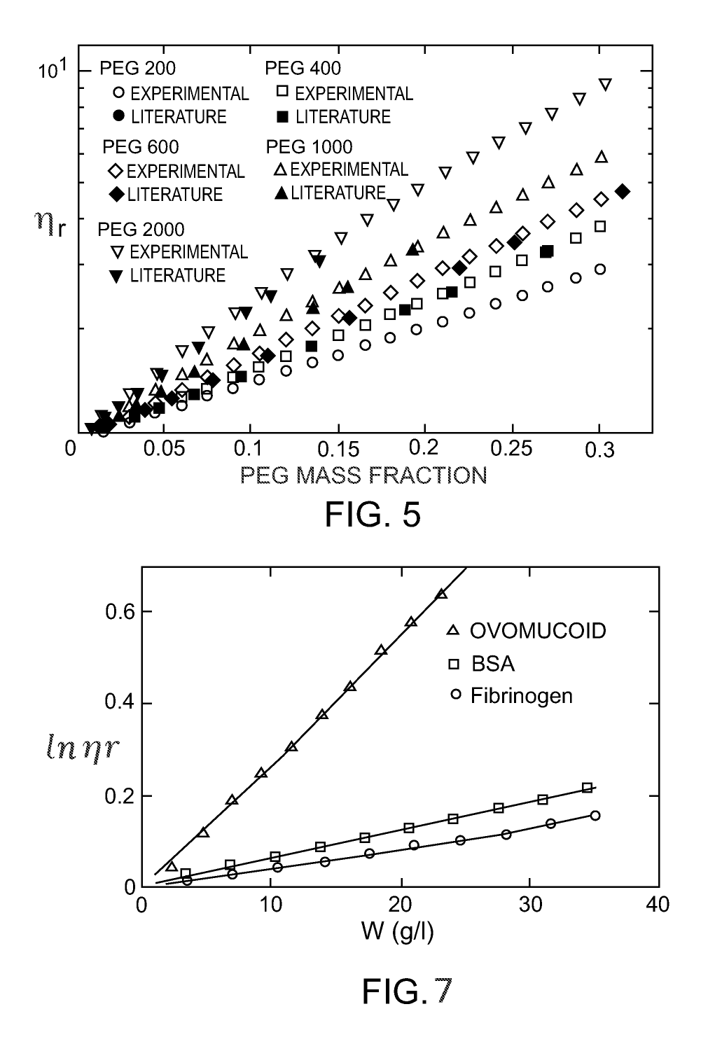

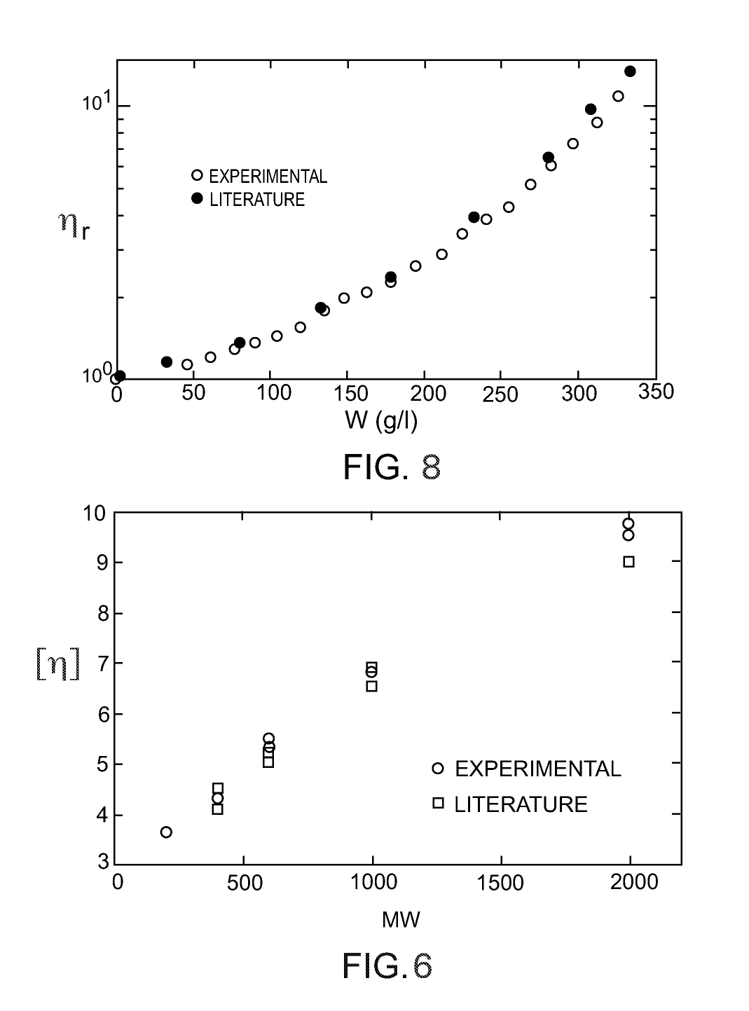

Measuring the viscosity of highly viscous solvents requires the complete mixing of all mixture components and the ability to measure pressure over a broad range of concentrations. To test the system under these conditions, concentrated solutions of 70% w/w Sucrose and 90% w/w glycerol were prepared, and the viscosity of these solutions was measured as a function of concentration by automated sequential dilutions of 2% and 5%. The measured viscosities are plotted as a function of concentration (converted to w/w units) together with results taken from standard tables (FIG. 4) (Lide 2004). Viscosity of polyethylene glycol (PEG) solutions. The viscosity of solutions of several different size fractions of PEG in water at 25° C. was measured as a function of concentration in an automated series of dilutions from ˜30% w/v to ˜3% w/v. In FIG. 5, the results of measurements in our apparatus are compared with those of previous measurements (Kirincic and Klofutar 1999). The intrinsic viscosities were calculated with equation 8 and are in good agreement with the published data (FIG. 6).

Intrinsic Viscosity of Proteins.

The concentration dependence of the relative viscosity of solutions of ovomucoid, bovine serum albumin, and fibrinogen was measured via automated dilution in the low concentration regime. The results are plotted in FIG. 7 together with the respective best-fits of equation [8], yielding the estimates of the intrinsic viscosity of each protein listed in Table 2. Literature values are also tabulated for comparison.

TABLE 3
shows estimates of intrinsic viscosity of three
proteins measured at 25° C. Uncertainties indicated
correspond to ±1 standard error of estimate.

[η] (cm³/g)

| | Best fit of | | |
|---|---|---|---|
| | equation [8] | Literature | |
| Protein | to current data | data | References |
| ovomucoid | 6 (5.9-6.2) | 5.4-5.6 | (Donovan 1967, |
| | | | Waheed and |
| | | | Salahuddin 1975) |
| BSA | 3.6 (3.5-3.7) | 3.6-3.8 | (Buzzell and |
| | | | Tanford 1956)* |
| fibrinogen | 25 (23.3-26.5) | 25-34 | (Shulman 1953)* |

*includes data compilation from other works.

Viscosity of Hemoglobin Over a Broad Range of Concentration.

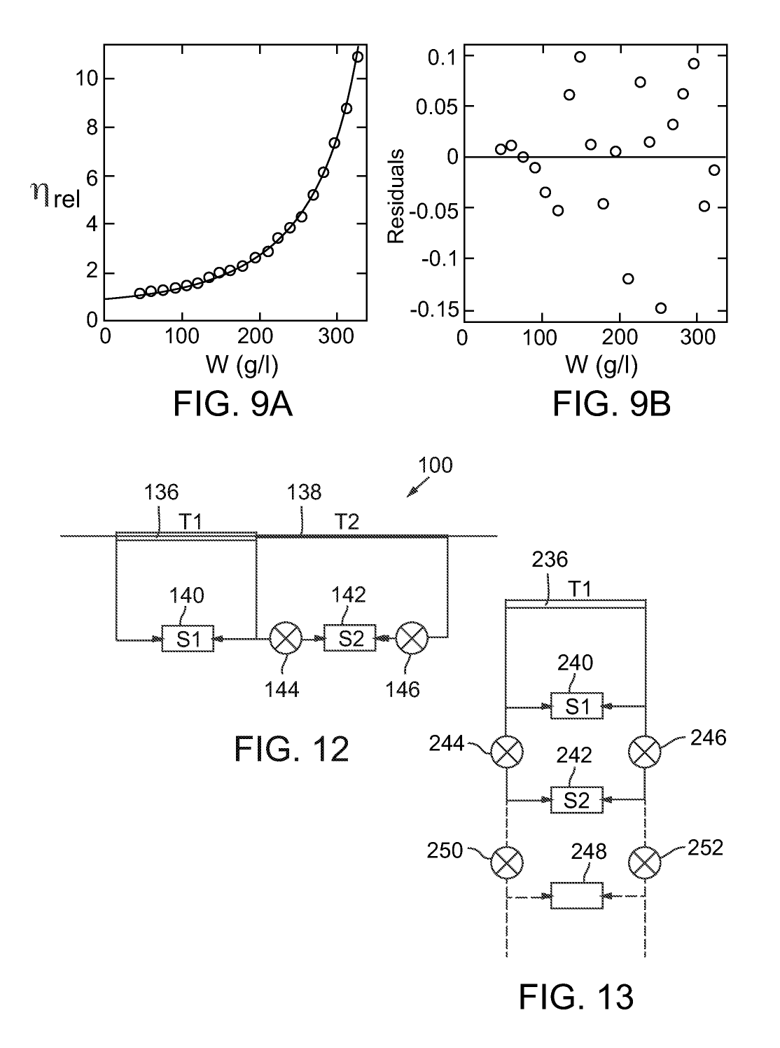

The concentration dependence of the viscosity of purified hemoglobin at 25° C. was measured by automated dilution of a solution initially containing 330 g/l protein. The concentration dependence of the viscosity was modeled with the generalized Mooney (Ross and Minton 1977).

$$η = {η}_{0} exp ( \frac{⌊ η ⌋ c}{1 - ( k / v ) [ η ] c} ) \qquad [ 9 ]$$

Where c is solute concentration, k is the crowding factor and is a shape factor for deviation from sphere. The results are plotted in FIG. 8 and compared with data published previously. When all parameters are free, the fit results in parameter values very close to the previously reported but with a relatively broad confidence limits. Fixing only [η] to 0.036 results in much narrower confidence limits for k/ν (FIG. 9A-9B). Fitting the concentration dependence of viscosity reported by Chien (Chien, Usami et al. 1970) to the model, gives the expected value of k/ν, but only if both η_{c} and [η] are fixed, as done previously by Minton (Ross and Minton 1977). The value for k/ν=0.43 was also obtained by Monkos (Monkos 1994).

### Discussion

Measurement of the rheological properties of concentrated protein solutions can be a challenging task with implications for pharmaceutical formulations and delivery of concentrated therapeutics, as well as for characterization and understanding of the non-ideal behavior of these complex macromolecules. With this in mind, in one embodiment, the present subject matter provides a viscometer/rheometer for automatically measuring the concentration and shear dependence of viscosity of a small total volume of solution over a broad range of viscosities (e.g., ˜1-1000 cP) and shear rates (e.g., 10¹-10³ s-1). The instrument was tested by measuring the concentration dependence of viscosity in solutions exhibiting both Newtonian and non-Newtonian behavior. As discussed above, results from an apparatus of the present subject matter was compared to previously published viscosity results and was found to be accurate in both the high and low viscosity regime.

A unique feature of one embodiment of the present subject matter is the automated dilution scheme, which is not generally available in any of the commercially available viscometers/rheometers. The concentration gradient can be created automatically by using a single syringe pump and a distribution valve (for example, a multi-way valve (e.g., 6-way)), permitting faster and more accurate dilutions than can be performed manually. This design not only permits the dilution of a single solute species, but can be extended to varying the composition of solutions containing multiple solute species, enabling, for example, a comprehensive study of the effect of varying a small solute on the viscosity of a solution of a macromolecule at constant concentration.

In one embodiment, automated dilution experiments may be carried out on solutions with a maximum viscosity of ˜10³ cP, and higher viscosities may be measured by direct injection.

In one example, a complete concentration gradient experiment including 20 dilution steps with three shear rates at each concentration can be carried out in about 1.5 hour, such as measuring a 350 mg/ml BSA solution at pH 7 and a viscosity of ˜40 cP.

In one embodiment, a total solution volume of <0.75 ml is sufficient to perform a 26 step dilution gradient, which is equivalent to ˜30 ul of solution per dilution, with a recovery yield of >95%.

In other embodiments, sample volume can be further reduced to ˜0.5 ml or even less than 0.5 ml. This is accomplished by using tubing having a 0.02 in. inner diameter. In addition, the position of the valve is changed to minimize the distances between the valve and the pump and between the valve and the water bath.

In contrast, many commercial viscometers/rheometers require sample volumes as large as 10-45 ml, and are hence not suitable for studies of concentrated solutions of proteins that might be conveniently available only in small quantities. Commercial instruments that allow for small sample volume do not allow for automated dilution of the initial sample volume. As a consequence, in order to achieve a concentration gradient over the range reported here, a substantially larger total sample volume is required. The cyclic fluid flow design of our apparatus permits us to make several replicated measurements on a sample to obtain a more precise measurement without adding material.

The ability to measure the viscosity over a range of flow rates, and hence shear rates, provides rheometric capability. Cone and plate rheometers, usually limited to shear rates of greater than ˜1000 s⁻¹, are prone to errors when measuring low viscosity protein solutions due to surface tension and evaporation of the sample. In contrast, some embodiments described herein allow for shear rates spanning three orders of magnitude (<10-˜5000 s-1) even for a very dilute solution.

Very low shear rates (<1 s⁻¹) can be accessible if pressure sensors having adequate pressure sensitivity are employed. Some inexpensive commercially available pressure sensors may not have adequate pressure sensitivity to measure very low shear rates (<1 s⁻¹).

Generally, a range of viscometer or rheometer measurements might be designed in existing instruments by using interchangeable accessories, such as various size balls for the falling ball viscometer, differently angled cones for the cone/plate, or sensor chips for the Rheosense VROC apparatus. In contrast, in some embodiments of the present subject matter, the range and resolution of measurement can be varied broadly by simple variation of capillary length and inner diameter. For example, the present subject matter may optionally use inexpensive PEEK™ tubing of various diameters and pressure sensors of various ranges, the replacement of which is quick and simple.

By measuring the pressure drop across a cylindrical capillary rather than a rectangular channel as in the Rheosense VROC, embodiments of the present subject matter can calculate viscosity in a straightforward fashion via Poiseuille's law rather than by recourse to non-analytical solutions of fluid flow that require extensive instrumental calibration to correct for nonlinear response. Calibration of embodiments of the present subject matter can show that they provide an accurate dilution scheme.

### User Interface

### Parameter Input Window

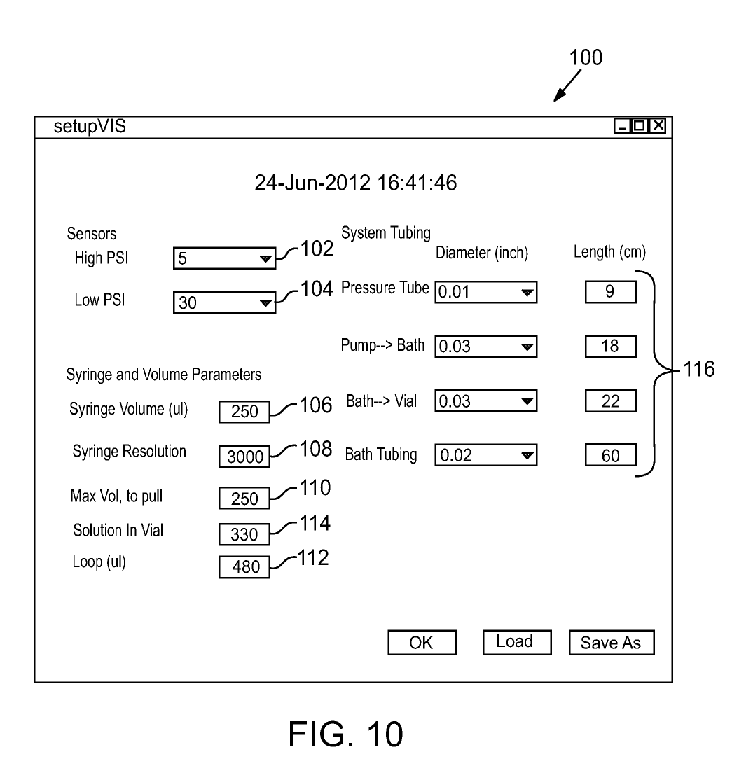

In one embodiment, while running the program, a parameter input window 100 will open (FIG. 10) in which the user specifies the system parameters: (1) the range 102, 104 of each of the two sensors (2) the syringe volume and pump step resolution 106, 108 (3) the maximum volume the syringe can pull without introducing air 110 (4) the volume 112 of the tubing in which the sample circulates and (5) solution volume 114 in the sample vial 20 and (6) the tubing diameter and length for different tubing parts 116. This data is stored and printed to the experiment report.

### Main User Interface

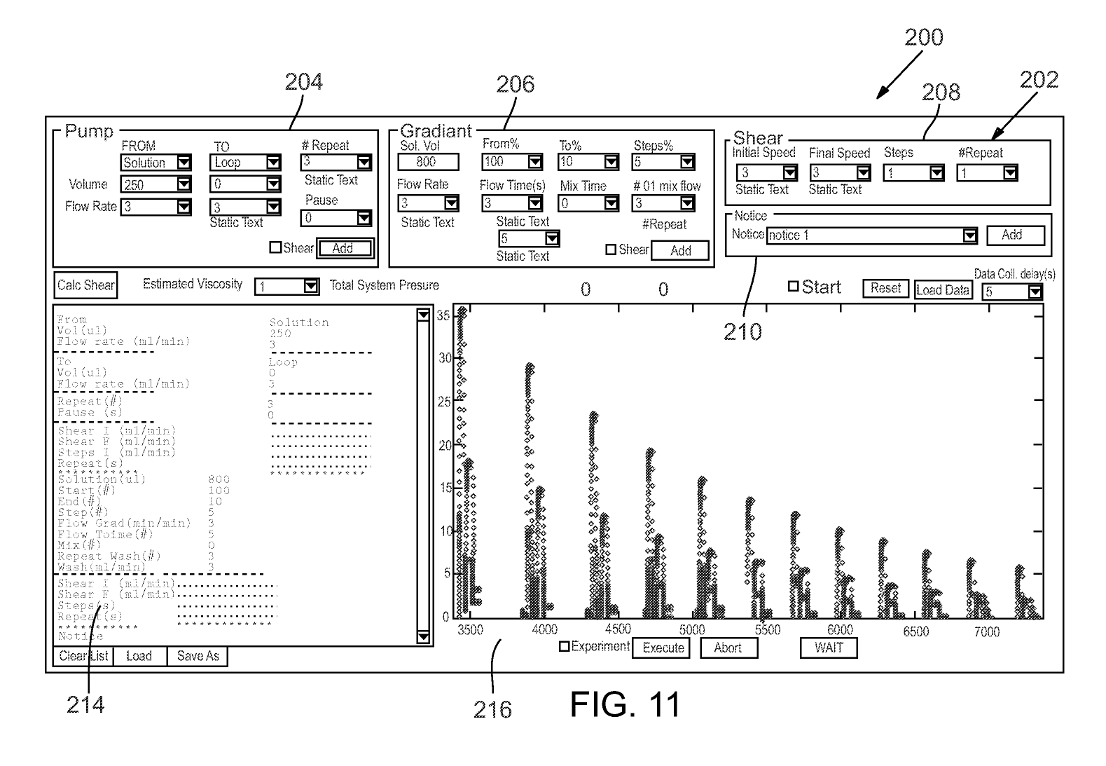

In another embodiment as shown in FIG. 11, an exemplary user interface 200 consists of three main parts.
1. A command builder 202.
   Appears as four border colored boxes in the upper part of the window. The different boxes allows the user to program a sequence of events for a specific experiment.
      Blue box (left) 204—syringe pull/dispense commands.
      Red box (middle) 206—Gradient builder.
      Green box (upper right) 208—Shear rate commands. Can be added to any step in a gradient or a pull/dispense sequence.
      Black box (lower right) 210—Pause and display reminder text. Enables a predefined pause of execution with a message box reminder of what to perform.
2. A command viewer 214 (Lower left)—the user can review the experiment command sequence. This data is stored and printed to the experiment report.
3. Sensor output graphic window 216 (Lower right)—displays real time pressure signal data acquisition.

### Example 2

### Denaturation by Heat or Addition of Chaotropic Additives

Protein denaturation is the process of a conformational transition from a compact, folded structure, to an ensemble of random coil conformations. Existing methods for protein denaturation employ the stepwise addition of chaotropic additives or a gradual increase in temperature. The change of the protein shape and size affects also the intrinsic viscosity of the protein, therefore the denaturation process can be monitored by viscosity measurements of the solution at different stages of the denaturation using devices of the present subject matter. For example, the temperature of the sample can be steadily increased or decreased and viscosity measurements can be made.

### Example 3

### Reversible and Irreversible Self-Association

Many colloidal suspensions of either biological or synthetic particles may undergo reversible or irreversible self-association under solution conditions that are usually solute specific. It has been shown that the solution viscosity correlates with the state of aggregation and can be used to estimate solution stability (Bohidar 1998, Saito, Hasegawa et al. 2012). The current invention may be used to study the stability of a colloidal suspension upon modulation of concentration, temperature, pH or cosolutes. Specifically, determination of the concentration dependence of viscosity for a colloidal quasispherical suspension at high concentration can potentially provide quantitative determination of the state of self-association using recently developed models (Minton 2012).

### Example 4

### Illustrative Simulations of Shear Rate Range

The following are illustrative simulations of the shear rate range for a pressure tubing of diameter D=0.01″ and length L=5 cm.

### Shear Rate at the Low Viscosity Range (1-10 cP)

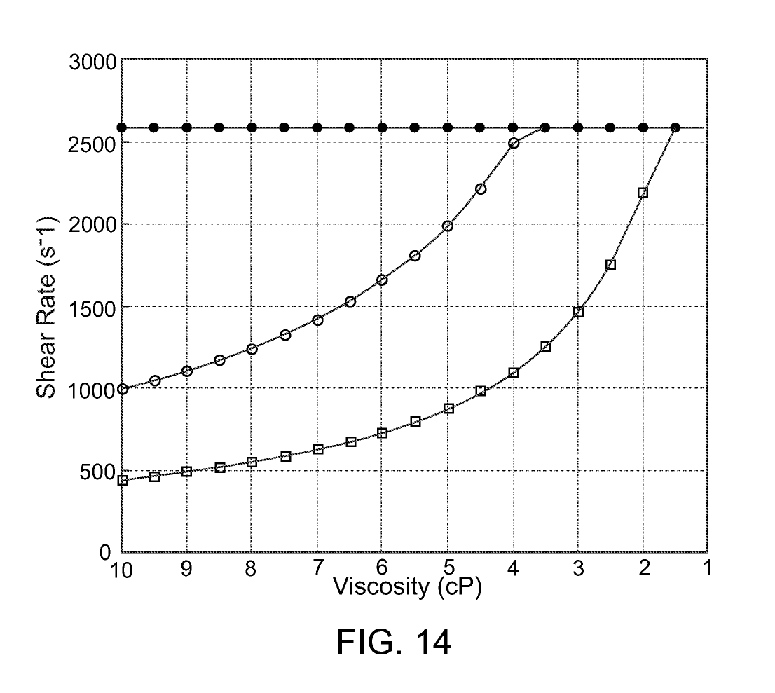

As shown in FIG. 14, the shear rate at the low viscosity range is plotted at three backpressure limits: 30 psi (square data points), 68 psi (circular data points), and 250 psi (filled circular data points). The lowest curve is the lower limit of the shear rate at each backpressure limit, which overlaps for all plots.

### Shear at the Mid Viscosity Range (1-150 cP)

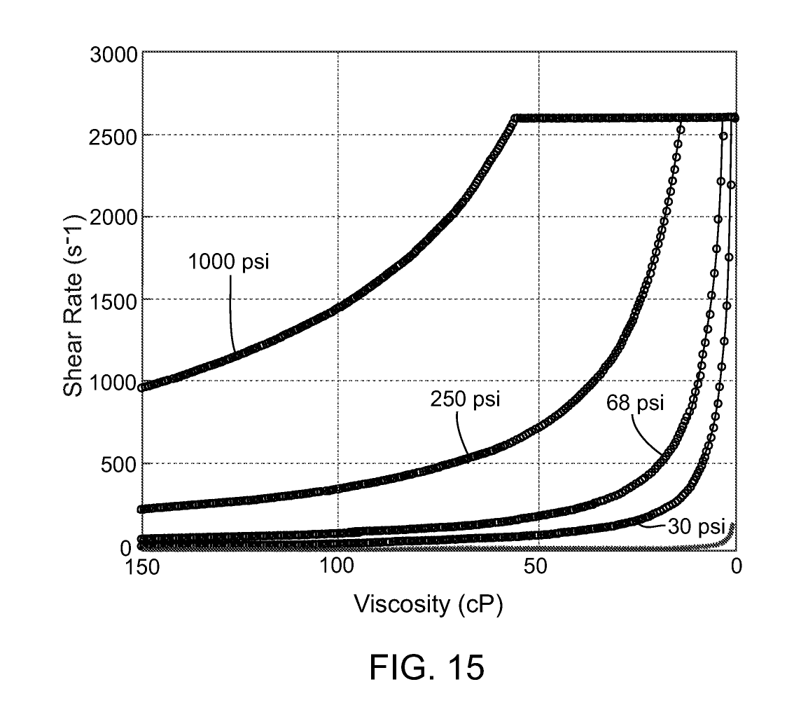

The midrange covers the normal working range for concentrated protein solutions in FIG. 15, the effects of increasing the backpressure limit are plotted for backpressures of 30 psi, 68 psi, 250 psi and 1000 psi as indicated in the figure. Distribution valves with a backpressure of 1000 psi are commercially available (and see below for an implementation using a valve with a backpressure rating of 6000 psi).

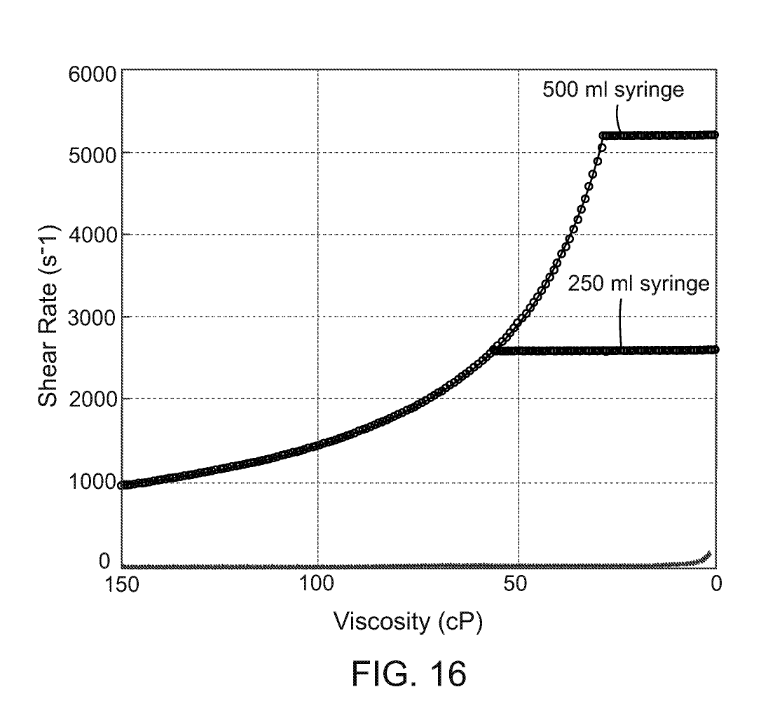

As shown in FIG. 16, increasing the volume in the syringe increases the range of flow rates and hence the range of shear rates. As one specific example, changing from a 250 ul syringe to a 500 ul syringe doubles the shear rate range.

### Shear at the High Viscosity Range (1-1000 cP)

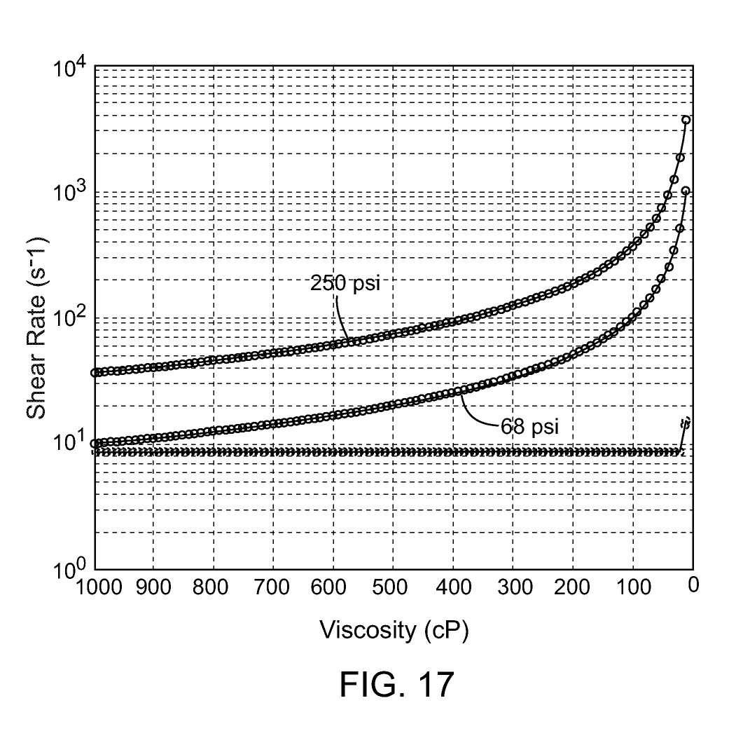

At very high viscosities (1000 psi), the shear range is very small. FIG. 17 shows the respective shear rate for a system with a backpressure of 68 psi and 250 psi as indicated.

An alternative configuration 100 for the tubing and pressure sensors is shown in FIG. 12. In FIG. 12, multiple tubing segments of different diameters and/or lengths are connected in series (two such segments 136, 138 are shown in the figure). Each segment is selected for desired sensitivity to specific viscosity and/or shear rate ranges. As shown, a first pressure sensor 140 is connected in parallel to the first segment 136 to measure a pressure change of a flow across the first segment 136. Similarly, a second sensor 142 is connected in parallel to the second segment 138 to measure a pressure change of a flow across the second segment 138. The valves 144, 146 may be manual or automatic valves. The valves 144, 146 are configured to prevent the sensors, which may be high-sensitivity pressure sensors, from experiencing pressures above their operating ranges.

FIG. 13 is an expanded view of a parallel pressure sensor and tubing arrangement similar to the FIG. 1A embodiment. In FIG. 13, the solution is conveyed through a single tubing segment 236 of a predetermined diameter and length. A low sensitivity pressure sensor 240 is connected to the tubing segment 236 directly in parallel to record high shear rates or high viscosity pressure differentials. A high-sensitivity pressure sensor 242 is connected in parallel to the segment 236, but manual or automatic valves 244, 246 are positioned as shown to protect the high-sensitivity pressure sensor 242 from over pressure. As indicated in dashed lines, it is possible to arrange a third pressure sensor 248 of even a higher sensitivity than the second pressure sensor 242, and to arrange valves 250, 252 to protect the third pressure sensor 248. Of course, even additional pressure sensors tailored for different ranges could be configured.

According to one implementation, an exemplary sample loading process includes the following acts:
1. Washing all of the system tubing with solution buffer.
2. Drying the closed-circuit tubings with compressed filtered air (syringe inlet at distribution valve to solution inlet and syringe inlet at distribution valve to solution outlet).
3. Loading the syringe with the sample, and keeping the remaining solution in the solution vial.
4. Connecting the vial cap to the solution vial.
5. Connecting of syringe to distribution valve and pushing the solution to solution vial through the solution inlet (clears air from tube of solution inlet).
6. Loading the syringe with sample solution.
7. Pushing the solution through the pressure tubing into the solution outlet
8. Repeating steps (6) and (7) until the system is loaded with the sample and the sample is mixed.
9. The method of the viscosity measurement is the same as described above (steps 1-8).

### Example 5

In one implementation, the instrument was upgraded to increase the range of viscosity and shear rate measurements by replacing a low pressure valve with a backpressure rating of 100 psi with a high pressure distribution valve having a backpressure rating of 6000 psi. A Rheodyne TitanIID valve is one suitable valve having a backpressure rating of 6000 psi. As a result, increases in the ranges of viscosities and shear rates with which the instrument can be used were observed.

Specifically, using a valve with a backpressure rating of 6000 psi allows using the full lower backpressure rating of other components. For example, a Hamilton 250 microliter syringe has a backpressure of 500 psig and a Hamilton 500 microliter syringe has a backpressure rating of 700 psig. Therefore, with a valve having a high backpressure rating such as 6000 psi, either syringe can be used to measure viscosities at shear rates producing a differential pressure of at least about 250 psi.

### Example 6

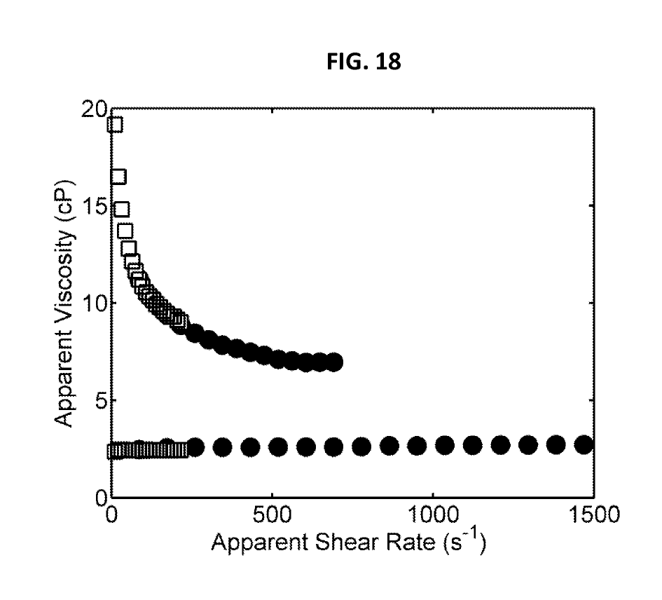

In FIG. 18, the measured dependence of viscosity upon shear rate at 20° C. is plotted 18 for a 28% (w/w) sucrose solution (lower curve), exhibiting Newtonian behavior, and for a 1% (w/w) PEG solution (˜10⁶ Da)(upper curve), exhibiting non-Newtonian shear thinning behavior in agreement with published results. In order to cover a wide range of shear rates, the viscosity of each solution was measured using the instrument with two different sizes of pressure tubing at changing flow rates. Specifically, measurements were taken with a first pressure tubing having a 0.01 in. inner diameter and a 4.4 cm length (data points are solid circles) and with a second pressure tubing having a 0.02 in. inner diameter and a 20 cm length (data points are hollow squares), at flow rates ranging from 10 to 0.5 mL/min. In the case of the non-Newtonian behavior, the calculated values for shear rate and viscosity are apparent values and may be corrected to true values by application of the Rabinowitsch equation.

### Example 7

In one exemplary embodiment, a total solution volume of less than about 0.5 ml is sufficient to perform a multi-step dilution gradient. This is accomplished, e.g., by using tubing having a 0.02 in. inner diameter. In addition, the position of the valve is changed to minimize the distances between the valve and the pump and between the valve and the water bath.

### Additional Advantages

Among other benefits, implementations of the instrument allow sample volumes to be reduced, which is especially important when working with limited amounts of samples, such as in testing biopharmaceuticals in development stages. The instrument provides for achieving results quickly and in a reproducible manner.

In addition to protein-based drugs for the pharmaceutical industry, the instrument and methods have application in the ink and coatings (e.g., paint) industries in which the involved materials have high shear rates. In addition, the described approaches have application to the characterization of polymeric materials that flow at high temperatures and solidify upon cooling, such as are used in 3D printing.

Among other experimental uses, the described approaches can be used to study the dependence of the viscosity of concentrated therapeutic monoclonal antibodies upon concentration and shear rate.

### ALTERNATIVES

The technologies from any example can be combined with the technologies described in any one or more of the other examples. In view of the many possible embodiments to which the principles of the disclosed technology may be applied, it should be recognized that the illustrated embodiments are examples of the disclosed technology and should not be taken as a limitation on the scope of the disclosed technology. Rather, the scope of the disclosed technology includes what is covered by the following claims. We therefore claim as our invention all that comes within the scope and spirit of the claims.

1. An automated viscometer, comprising:
a closed-circuit pressure tubing system through which a viscosity sample can flow;
at least one in-line pump for urging the sample through the tubing system;
at least one in-line distribution valve connected to the at least one in-line pump for adding diluting liquid or sample to the tubing system, removing diluting liquid or sample from the tubing system, or combinations thereof;
at least one in-line pressure test-zone tubing section; and
at least one pressure differential sensor for measuring the change in pressure across the pressure test-zone tubing section.

2. The viscometer of claim 1, further comprising at least one in-line sample reservoir from which sample can be fed to the pump and to which sample can be returned.

3. The viscometer of claim 1, wherein the at least one in-line distribution valve comprises at least one in-line multiple distribution valve programmable for automated directional pumping of diluting liquid, waste, sample, or combinations thereof.

4. The viscometer of claim 1, wherein the at least one distribution valve is connected to solvent inlet or solvent reservoir, a waste outlet or waste reservoir, an in-line sample reservoir, or a combination thereof.

5. The viscometer of claim 1, wherein the at least one pressure differential sensor comprises two or more pressure differential sensors connected in parallel, in series, or in a combination thereof.

6. The viscometer of claim 5, wherein the first pressure differential sensor is isolated from the second pressure differential sensor by at least one pressure valve on each of two sides of the second pressure differential sensor.

7. The viscometer of claim 5, wherein the first pressure differential sensor senses a higher pressure range than a pressure range sensed by the second pressure differential sensor.

8. The viscometer of claim 5, wherein the first pressure differential sensor senses pressure ranging from about 5 psi to about 250 psi, and the second pressure differential sensor senses pressure ranging from about 1 psi to about 5 psi.

9. The viscometer of claim 1, wherein a minimum sample size is about 1 mL or less.

10. The viscometer of claim 1, further comprising a thermostatic control device.

11. The viscometer of claim 1, further comprising a coiled section of the closed-circuit pressure tubing for use in combination with a thermostatic control device.

12. The viscometer of claim 1, further comprising programmable controls for automated control of the at least one in-line pump for moving the sample through the closed-circuit tubing system, through the at least one distribution valve, or combinations thereof.

13. The viscometer of claim 1, further comprising programmable controls for automated control of the at least one distribution valve for moving sample through the closed-circuit tubing system, for adding liquid or sample to the closed-circuit tubing system, for removing liquid or sample from the closed-circuit tubing system, for diluting each successive sample with a defined serial dilution, or combinations thereof.

14. The viscometer of claim 1, further comprising a data acquisition module connected to the at least one pressure differential sensor for acquiring and storing pressure differential sensor data.

15. The viscometer of claim 1, further comprising:
(a) programmable controls for automated control of the at least one in-line pump for moving the sample through the closed-circuit tubing system, for moving the sample through the at least one distribution valve, or combinations thereof;
(b) programmable controls for automated control of the at least one distribution valve for moving sample through the closed-circuit tubing system, for adding diluting liquid or sample to the closed-circuit tubing system, for removing diluting liquid or sample from the closed-circuit tubing system, for diluting each successive sample with a predetermined serial dilution, or combinations thereof;
(c) a data acquisition module connected to the at least one pressure differential sensors for acquiring and storing pressure differential sensor data; and
(d) a programmable system control module connected to (a), (b), and (c).

16. The viscometer of claim 1, wherein the pressure test-zone tubing section has a defined length and cross-sectional area.

17. The viscometer of claim 1, further comprising an in-line sample flow cell for observing physical properties of the sample.

18. The viscometer of claim 17, wherein the in-line sample flow cell is coupled to a device for observing physical properties of the sample selected from the group consisting of light scattering, light absorbance, fluorescence, NMR, ESR, Raman spectra.

19. A method of measuring viscosity as a function of concentration and shear dependence, comprising:
(a) injecting a small volume sample at a known concentration and flow rate through a pressure tubing system;
(b) recording measurements from at least one low sensitivity sensor connected to measure pressure in the pressure tubing system
(c) collecting the sample in an in-line sample reservoir arranged in-line with the pressure tubing system;
(d) diluting the sample in the sample reservoir by removing a predetermined amount of sample from the sample reservoir through a distribution valve and adding a predetermined amount of a diluent provided through a distribution valve;
(e) circulating the diluted sample through the pressure tubing system;
(f) measuring the pressure at the pressure sensor to determine a pressure measurement corresponding to a current dilution.

20. The method of claim 19, wherein the steps (c), (d), (e) and (f) are repeated until the current dilution reaches a desired minimum dilution level.

21. The method of claim 19, wherein the steps (a), (b, (c), (d), (e) and (f) are repeated for a different flow rate.
